(19) United States
(12) Patent Application Publication
(10) Pub. No.: US 2007/0174085 A1
(43) Pub. Date: Jul. 26, 2007

(54) SYSTEM AND METHOD FOR ORDERED RECOMMENDATION OF HEALTHCARE OR PERSONAL CARE PRODUCTS

(76) Inventors: Charles C. Koo, Palo Alto, CA (US); Dennis Wu, Palo Alto, CA (US); Roger Bertman, Los Altos, CA (US)

Correspondence Address: SQUIRE, SANDERS & DEMPSEY L.L.P, PATENT DEPARTMENT, ONE MARITIME PLAZA, SUITE 300, SAN FRANCISCO, CA, 94111-3492, US

(21) Appl. No.: 11/462,043

(22) Filed: Aug. 3, 2006

Related U.S. Application Data
(60) Provisional application No. 60/762,792, filed on Jan. 26, 2006.

(51) Int. Cl. G06Q 50/00

(52) U.S. Cl. 705/2

(57) ABSTRACT

A system and method rank search results based on bidding for search result rankings. The search result rankings can include healthcare products based on search terms of symptoms.

### PRIORITY REFERENCE TO PRIOR APPLICATION

This application claims benefit of and incorporates by reference U.S. Patent Application No. 60/762,792, filed on Jan. 26, 2006, by inventors Charles C. Koo et al. This application is also related to and incorporates by reference U.S. patent application Ser. No. 11/315,410 filed on Dec. 22, 2005.

### TECHNICAL FIELD

This invention relates generally to healthcare/personal care products (pharmaceuticals, vitamins, over the counter medications, skin care, etc.), and more particularly, but not exclusively, provides a system and method for the ordered recommendation of healthcare/personal care products based, at least in part, on bidding for placement.

### BACKGROUND

A retail store or a pharmacy has tens of thousands of products on the shelf, many of which are complex and require advice from experts. In some cases, consumers do not know the product but only know the problem that they want to solve. For example, there are more than two thousand over-the-counter (OTC) drug products for the treatment of a variety of symptoms and conditions. Consumers entering the store to find the right product to treat their particular symptoms and conditions are faced with a confusing set of choices, and they are ill-equipped and uncertain of how to make the right choice to meet their needs. Faced with this uncertainty, they typically spend a good deal of time, ranging from 10 to 20 minutes, comparing packages trying to understand the ingredients of each product and how those ingredients relate to the particular symptoms they want to relieve, diseases or conditions they have, or other considerations such as age or allergies. Their intent is to find the right product which has all of the ingredients they need with no ingredients that they don't need or their conditions prohibit. Frequently, after searching on their own, consumers ask a pharmacist for advice. Similar problem exists in most health-and-beauty products such as vitamins, supplements and cosmetics (including skin care products).

From the retailer and/or manufacturers' perspective, marketing products to consumers in the face of this confusion has always been a difficult challenge. Manufacturers want to position their products to meet as many consumer symptom needs as possible and to differentiate them from competitors' products with similar effects. Manufacturers spend millions of dollars to package, advertise, and promote their products in their attempt to maximize their share of product sales.

As such, a new system and method are needed that enable healthcare product recommendation combined with consumer marketing.

### SUMMARY

The present invention provides a system and method for the bid-based ranking of search results, in particular, of healthcare products selected based on symptoms or problems and optionally constraints.

In an embodiment of the invention, the method comprises: receiving a (set of) symptom(s) or problem; determining healthcare products or solutions for treating the symptom(s) or problem, respectively; ranking the determined products or solutions based on bids; and displaying the determined products or solutions based on the ranking.

In an embodiment of the invention, the system comprises: a graphical user interface, a search agent, and a ranking engine. The GUI receives at least one symptom or problem. The search agent, which is communicatively coupled to the GUI, determines healthcare products or solutions for treating the symptom(s) or problem, respectively. The ranking engine, which is communicatively coupled to the search agent, ranks the determined products or solutions based on bids. The GUI also displays the determined products or solutions based on the ranking.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1A is a block diagram illustrating a network in accordance with an embodiment of the invention;

FIG. 1B is a diagram illustrating a kiosk;

FIG. 2 is a block diagram illustrating components of the kiosk of FIGS. 1A and 1B;

FIG. 3 is a block diagram illustrating a persistent memory of a bid system of the network of FIG. 1A;

FIG. 4 is a block diagram illustrating a persistent memory of the kiosk;

FIG. 5 is a block diagram illustrating an “intent” graph;

FIG. 6 is a flowchart illustrating a method of searching;

FIG. 7 is a flowchart illustrating a method of displaying ranked healthcare products; and

FIG. 8 is a flowchart illustrating a method of determining highest bidders.

### DETAILED DESCRIPTION OF THE ILLUSTRATED EMBODIMENTS

The following description is provided to enable any person having ordinary skill in the art to make and use the invention, and is provided in the context of a particular application and its requirements. Various modifications to the embodiments will be readily apparent to those skilled in the art, and the principles defined herein may be applied to other embodiments and applications without departing from the spirit and scope of the invention. Thus, the present invention is not intended to be limited to the embodiments shown, but is to be accorded the widest scope consistent with the principles, features and teachings disclosed herein.

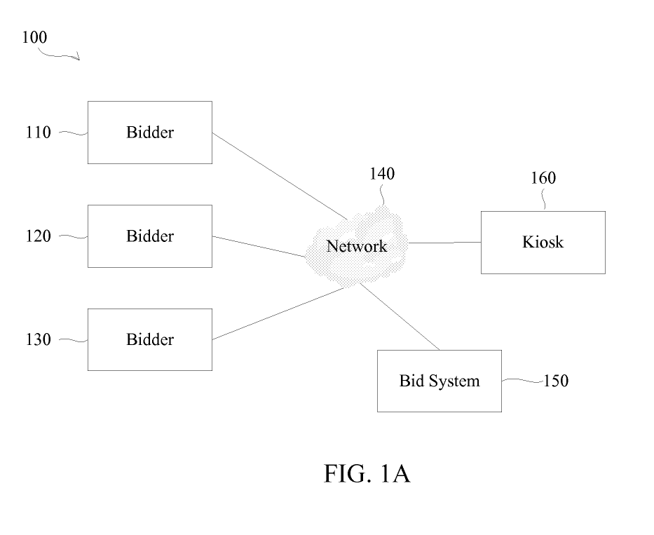

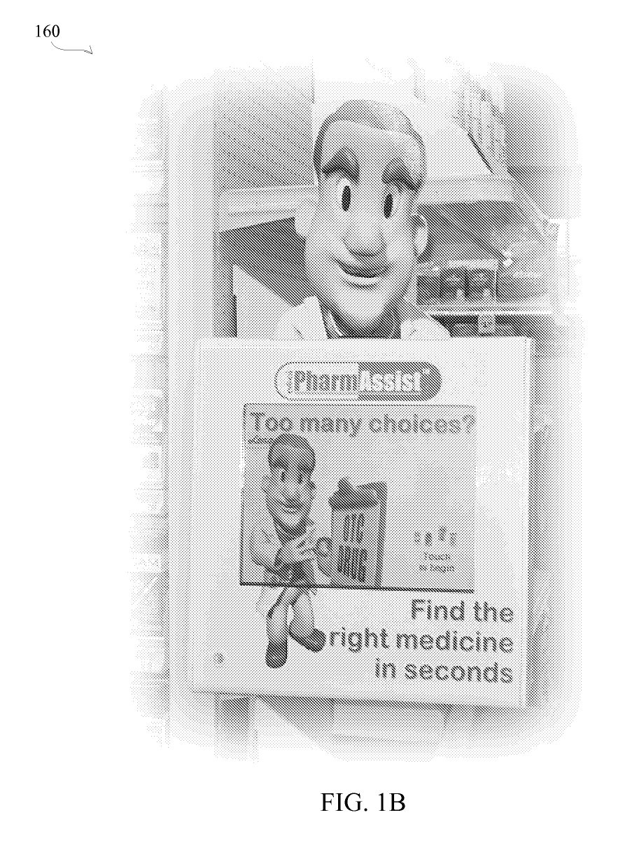

FIG. 1A is a block diagram illustrating a network 100 in accordance with an embodiment of the invention. The network 100 comprises a plurality of bidders, e.g., bidders 110, 120, and 130, communicatively coupled to a network 140, such as the Internet. Also coupled to the network 140 are a bid system 150 and a kiosk 160. In an embodiment of the invention, a plurality of kiosks 160 are communicatively coupled to the network 140. However, for ease of explanation, only a single kiosk 160 is illustrated. The plurality of kiosks can be located in drug stores or any other location where healthcare products are sold. One store can have multiple kiosks in an embodiment to ensure that consumers can easily find one and that one is always available for use.

The kiosk 160 implements a process which recommends the correct products to an individual consumer within a retailing/pharmacy environment for the problem (e.g., relieve a set of symptoms and conditions) specified by the consumer and presents the recommended products in a sequence based on, at least partially, fees paid by the manufacturers or others (e.g., advertising agents of the manufacturer; reseller; distributor; etc.) of the products.

The kiosk 160 uses search algorithms, e.g., deductive search algorithms, to identify all healthcare products (e.g., OTC drug products) that, in one embodiment, contain the minimum active ingredients to address the symptoms and conditions specified by a consumer. The deductive search algorithms dynamically narrow the universe of potential results as the user specifies symptoms and conditions. Interactions between the search algorithms and a knowledge base recognize symptoms and conditions that are causal or mutually exclusive. As the set of symptoms is specified, other symptoms or conditions that can not coexist in a diagnosis or recommendation are eliminated. Similarly, in one embodiment, as conditions are specified which preclude the use of certain active ingredients, those active ingredients are removed from the list of potential remedies. These algorithms result in a dynamic, real-time identification of possible diseases and treatments. Once the minimal list of active ingredients has been determined through this search algorithm, that list is used to determine all healthcare products that contain those active ingredients. This result meets the criterion of treating all specified symptoms within the constraints of the specified conditions. In an embodiment of the invention, the minimal active ingredients need not considered in product selection.

The final step is to “filter” out all healthcare/personal care products that contain any active ingredients other than those in the minimal list. This step assures that the consumer is recommended only the products which contain only the precise ingredients required to treat the specified symptoms recognizing the specified condition constraints.

Once the set of recommended healthcare products that, in one embodiment, have the minimal set of ingredients and meeting all condition constraints has been determined, the sequence and/or manner in which those products are presented is determined based on business parameters, such as fees paid by the manufacturers of those products or negotiated positions by the retailer, in order to accomplish their specific product advertising and/or promotion objectives. Because all of the recommended products have been specifically determined to meet the needs of the specific consumer within the store selling those products, there is substantial value to a manufacturer having its product presented in a differentiated fashion from that of competitors' products. (Note that, in one embodiment, because all of the recommended products are pharmacologically equivalent, the consumer is assured that all recommended products meet his/her needs.) Four types of differentiated presentation are possible; first, the presentation position(ranking or sequence) of the product, understanding that the earlier in the list a product is presented, the more likely it will be chosen by the consumer; second, a message banner can be displayed along with the product recommendation to reflect the manufacturer's message, such as new product, price reduction, etc.; third, a static or animated audio or visual message/commercial can be presented for a specific product in which a consumer has indicated interest by requesting further information from the system on that product; and fourth, a coupon conveying information and/or a discount offer can be printed in order to assist and/or motivate a consumer to buy the recommended product.

In an embodiment of the invention, in addition to or in place of the kiosk 160, a web server can communicate with users coupled to the network 140 and provide the functionality of the kiosk 160 without the need for a user to be physically in a store. For example, the functionality of the kiosk can be incorporated in an online healthcare product store. The kiosk 160 will be discussed in further detail below.

Bidders 110-130 communicate with the bid system 150 to bid for product placement. Generally, the higher the bid relative to other bids, the better the product placement. Bids can be for a group of products (e.g., a product cluster that includes set of products from a single bidder that have the same active ingredients and which vary in the number of dosages per package and/or certain packaging, form (capsule vs. liquid) or flavoring variations) or a single product, and bids can be restricted geographically and/or over time. For example, bidding for a topical hydrocortisone product that relives itching can be restricted to regions having mosquitoes over certain time periods (e.g., Alaska in the Summer and Florida year round). Other criteria can include demographics, per store, per region, per chain or nation-wide at any time (e.g., target a dozen stores for a limited time during a pilot product launch, or change advertisement contents/offered-fees at any time).The bidding system 150 accepts the bids and forwards the bids to the relevant kiosks 160. The bidding system 150 also keeps track of ranked products (winning bids) at kiosks 160 so as to charge bidders accordingly. The bidding system 150 will be discussed in further detail below.

FIG. 1B is a diagram illustrating the kiosk 160. The kiosk 160 is designed to meet a number of criteria which enable it to effectively engage and interact with consumers and to operate efficiently in a retail store environment. These design criteria include the following:
Requires no floor space (“zero footprint”);
Occupies minimal shelf space;
Is fully self-contained (i.e. includes CPU, touch screen, power supply, speaker system, motion detectors, etc., and requires no integration with retailer IT systems);
“Plug and play” installation;
Remote management and support;

In addition to meeting these criteria, the kiosk also has a “Trade Dress” which provides an attractive, engaging presence for the consumer.

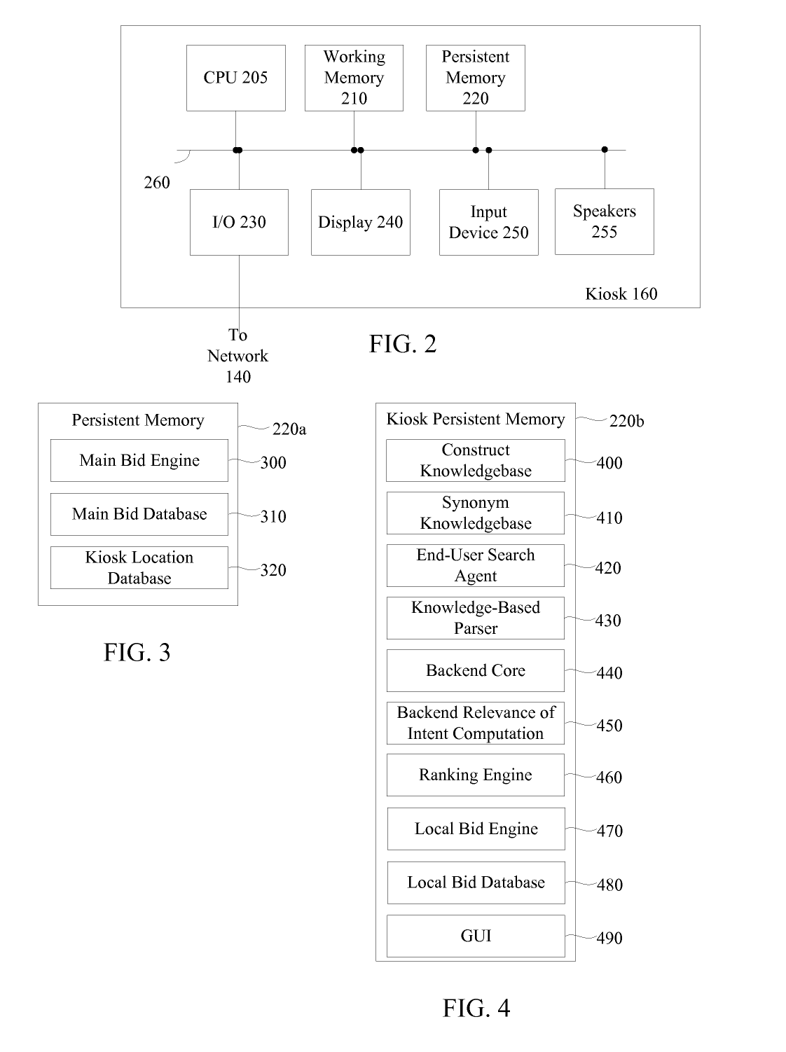

FIG. 2 is a block diagram illustrating components of the kiosk 160. In an embodiment of the invention, the bid system 150 can have substantially similar components. The kiosk 160 includes a central processing unit (CPU) 205; working memory 210; persistent memory 220; input/output (I/O) interface 230; display 240; input device 250; and speakers 255 all communicatively coupled to each other via a bus 260. The CPU 205 may include an INTEL PENTIUM microprocessor, a Motorola POWERPC microprocessor, or any other processor capable to execute software stored in the persistent memory 220. The working memory 210 may include random access memory (RAM) or any other type of read/write memory devices or combination of memory devices. The persistent memory 220 may include a hard drive, read only memory (ROM) or any other type of memory device or combination of memory devices that can retain data after the kiosk 160 is shut off. The I/O interface 230 is communicatively coupled, via wired or wireless techniques, to the network 140. The display 240 may include a flat panel display, cathode ray tube display, or any other display device. The input device 250, may include a keyboard, mouse, touch screen or other device for inputting data, or a combination of devices for inputting data. The speakers 255, which are optional like other components of the invention, emit sound in mono or stereo.

In an embodiment of the invention, the kiosk 160 may also include additional devices, such as network connections, additional memory, additional processors, LANs, input/output lines for transferring information across a hardware channel, the Internet or an intranet, etc. In an embodiment of the invention, the kiosk 160 includes a motion sensor coupled to the bus 260 that activates the display 240 and speakers 255, if any. One skilled in the art will also recognize that the programs and data may be received by and stored in the kiosk 160 in alternative ways. Further, in an embodiment of the invention, an ASIC is used in placed of the kiosk 160.

FIG. 3 is a block diagram illustrating a persistent memory 220a of the bid system 150. The memory 220a comprises a main bid engine 300; a main bid database 310; and a kiosk location database 320. The main bid engine 300 accepts bids from the bidders 110-130 for healthcare products (e.g., search results, not search terms). Bids can be blind (e.g., a bidder doesn't know if a product received any bids and if so, how much was bid); partially blind (e.g., a list of bidders and the order of bidders, but not amounts); or transparent (e.g., highest bid listed or all bids listed). Bids can be limited by time and/or geography. Bids can also be fixed or variable, e.g., equal to the highest bidder plus an increment. Bidders could be billed based on the ranked display and/or click through if a consumer wanted more information about the product.

Once a bid is accepted by the main bid engine 300, the main bid engine 300 stores the bid in the database 310 and transmits the bid to relevant kiosks as indicated in the kiosk location database 320. In an embodiment of the invention, each kiosk 160 then determines the ranking of received bids, as will be described further below. In another embodiment, the main bid engine 300 can determine the ranking of bids and transmit product placement information to the relevant kiosks.

FIG. 4 is a block diagram illustrating a persistent memory 220b of the kiosk 160. The persistent memory 220b includes a construct knowledgebase 400; a synonym knowledgebase 410; an end-user search agent 420; a knowledge-based parser 430; a backend core 440; and a backend relevance of intent computation engine 450. Further details are included in Table I, below.

In an embodiment of the invention knowledgebase comprises two major categories of knowledge: medical diagnosis and pharmacological knowledge; and OTC medicine knowledge.

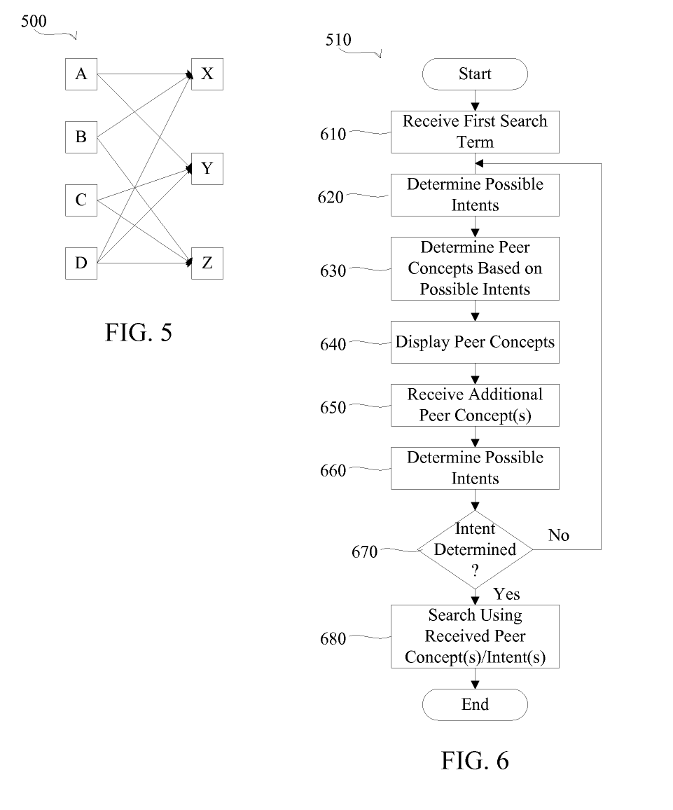

All data are integrated together to create a unified internal data structure which can be considered a concept graph (a.k.a. ontology), e.g., see FIG. 5, which are used by the search algorithms to answer end-user queries quickly.

Medical Domain Knowledge contains the following types of information: Symptoms; Diseases/medical conditions; Age group: “Adult”, “Child” and “Infant;” Active ingredients; Special group: risk group; and Side effects

There are also “relationships” built among concepts, an example of which is a comprehensive “causality relationship network” among all symptoms and diseases. This empowers a diagnosis to be determined based on symptoms and conditions.

The OTC medicine knowledge of the knowledgebase 400 contains data for all OTC medicines for the product categories and store environment in which the system is used. The following attributes define an OTC drug: Name; Active ingredient(s); Special considerations; Applicable age group(s); and Side effect(s).

In an embodiment of the invention, the persistent memory 220b can include other or additional software using different algorithms to perform searches for healthcare products. In an embodiment of the invention, searches are not limited to healthcare products.

```
TABLE I
Construct Knowledgebase
  Knowledge structure/construct
    Characteristic mapping (Attributes, taxonomy). For example:
      Concepts: cough
        Is-a: symptom
        ITD: allergy, asthma, COPD, bronchitis
      Concepts: allergy
        Is-a: disease
        DF: cough, wheezing, shortness-of-breath
        ITD: Claritin
      Concepts: Claritin
        Is-a: OTC medicine
        DF: allergy, allergic rhinitis, etc.
Synonym knowledgebase (For example:
  “Shortness of breath” is-a-synonym-of “breathlessness”
(strength = 1.0, which means they mean exactly the same.)
  “Hard to breath” is-a-synonym-of “breathlessness” (strength = 0.8)
End-user search agent (A program)
  UI (auto display of peer terms)
  UI (auto contraction by sets)
  UI (auto expansion for multiple intents/threads)
  UI (auto display of possible diseases)
  interface with the “relevance” count
Knowledge-based Parser (A program)
  map entered words to controlled words
  map controlled words to Concept Constructs based
  on the synonym knowledge base
Backend Core
  The Intent graph (dynamically constructed)
    Connect possible intents (Diagnosis CC)
    Calculate “Relevance Score” of each intent
  Relevance Score Calculation module
    Compute score based on Bayesian network
    Pre-compute scores based on Bayesian network
    Cache and index all possible scores
Backend “relevance” of intent computation
  Bayesian Prior from the counts
  Bayesian Posterior
```

The persistent memory 220b also includes a ranking engine 460, a local bid engine 470; a local bid database 480; and a graphical user interface (GUI) 490. The GUI 490 accepts search terms and displays search results. Once search results are determined, the ranking engine 460 ranks the search results (e.g., healthcare products) and the GUI 490 displays them based on determinations made by the local bid engine 470.

The GUI 490 attract consumer attention; enables non computer literate consumers to easily interact with the kiosk 160; presents products in strict accordance to the manufacturers' packaging; present products in a manner that is consistent with consumer expectations; and provides complete product advice within seconds.

The GUI 490 employs the following techniques: touch screen interface; motion detection; audio instruction; color graphics; images of product packaging, including “Drug Facts” on all sides of packages; and Virtual Shelf” product presentation.

The touch screen interface enables rapid, intuitive interaction without the use of a keyboard, thereby accommodating consumers who are not versed in using computer keyboards and the presentation of questions in a “multiple choice” fashion, thus minimizing the consumer's role in specifying symptoms and conditions. Motion detection enables the system to attract the consumer's attention by “speaking” as the consumer approaches. Audio instructions compliment the graphic display instructions to accommodate those who are more comfortable with verbal than written communications. The color graphics, particularly full color images of product packages, present a “virtual shelf” experience in which the consumer is presented products on the screen in much the same way they are presented on the shelf, except that only those products that meet their needs are presented. Then, as the consumer touches product images, the product package is “virtually rotated”, again in much the same way a consumer would rotate an actual product package. This experience enables even those consumers who are not versed in using computers to step through the product recommendation and selection process in a manner that is familiar and intuitive.

In an embodiment of the invention, the GUI 490 text descriptions for products will be listed in a random order if the recommended products have no bids. A color image of a product can be displayed if a bid has been placed for it. In an embodiment, up to four slots are available for images of products and the order from left to right in which they are presented can be based on the bid amount. The image can include a color image of the face of the product package, plus the ability for the consumer to see images of the other sides of the product. This presentation enables the consumer to read an enlarged image of the drug facts and other information which the bidder has provided on all sides of the package to inform and to convey key messages to the consumer. The consumer views the images by touching the image of the face of the product on the kiosk 160 touch screen 240 and then touching images of subsequent package sides on subsequent screen pages. In an embodiment, the clicking through to see other images of the product can be charged to the bidder (e.g., at 50% of the bid for ranked display). In an embodiment, a video can be shown when a consumer clicks through for an additional fee (e.g., twice the bid fee).

The local bid engine 470 determines the bids for the search results by looking up the bids in the local bid database 480. The local bid engine 470 can also calculate bids if bids are variable (e.g., a bidder can bid a variable bid equal to the highest bidder plus an increment, up to a maximum). If bids are time constrained, the bid engine 470 will include them if appropriate.

In an embodiment of the invention, the software in the persistent memory 220b can be resident in persistent memory 220a of the bid system 150 instead. As such, the kiosk 160 would then act as a “dumb terminal.”

FIG. 5 is a block diagram illustrating an intent graph 500. The graph 500 explains the concept behind the ontological searching method described herein. The graph 500 indicates search terms A, B, C, D and related intents X, Y, and Z. A intends-to-derive (ITD) X or Y; B ITD X or Z; C ITD Y or Z; and D ITD X or Z. The kiosk 160 can then determine peer concepts (search terms) associated with X and Y and display them (e.g., A, B, C, and D). The user's subsequent selection of a peer concept will narrow down the possible intents. For example, the selection of B ITD the intent of X only and the elimination of Y. In an embodiment of the invention, it is possible to have two intents simultaneously (e.g., a person could have symptoms of two different diseases indicating that he/she has two different diseases). In an embodiment of the invention, the intent for symptoms can also be a treatment or over-the-counter medicine for the symptoms, e.g., for the symptom headache, the intent is aspirin.

The “derived from” (DF) relations allow the user to select an intent and conversely narrows the selectable choices of the search terms for the user. The combination and iteration of ITDs and DFs substantially reduce the computation and formulate a refined query, and thus search results rapidly.

FIG. 6 is a flowchart illustrating a method 600 of searching. In an embodiment of the invention, the kiosk 160 performs the method 600. First, a search term (e.g., symptom) is received (610). Possible intents (disease diagnosis) are then determined (620). Then possible search terms are determined (630) and displayed (640) based on possible intents. A user then selects one or more additional search terms, which are received (650) and possible intents are then determined (660). Due to the receipt of additional search terms, the intent may be determined as discussed above in conjunction with FIG. 5. If the intent is (670) determined or there are no more search terms, then a search is performed (680) based on intent(s) and/or search term(s) selected by the user and received. In an embodiment, the method 600 can include transmitting the search term(s) and/or intent(s) to a search engine to perform the search instead of the performing (680). The method 600 then ends. Otherwise, the method 600 repeats from (620). In an embodiment of the invention, the method 600 can be halted at any point and the search performed (680) using any received search term(s) and/or intent(s). In an embodiment of the invention, the method 600 also includes constraints in the search based on limitations entered by a consumer (e.g., if the consumer indicates an allergy to an antibiotic, any product having that antibiotic will be excluded from search results). Constraints/limitations can be based on allergies, age, dietary restrictions, and/other factors. In an embodiment of the invention, other search methods can be used to determine relevant healthcare products.

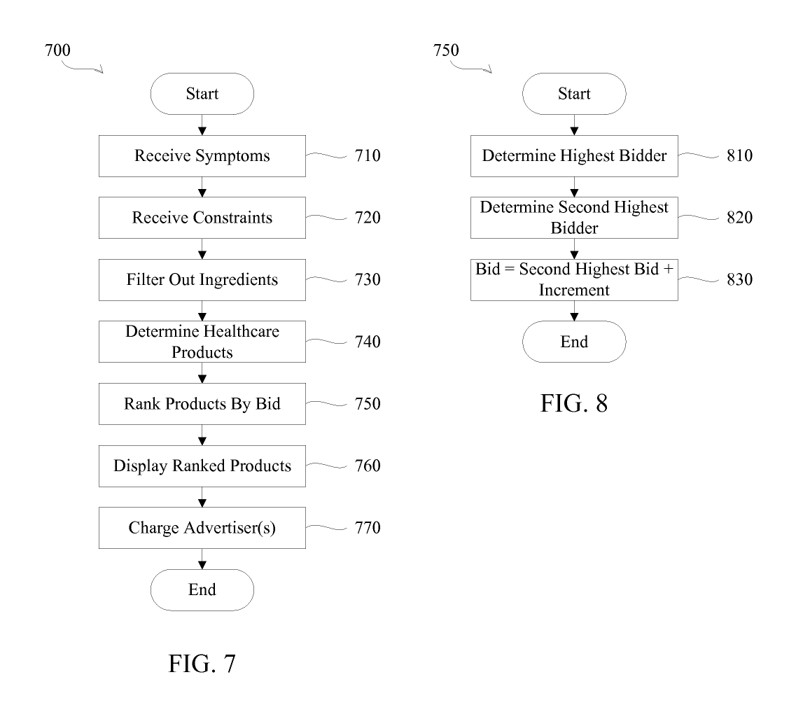

FIG. 7 is a flowchart illustrating a method 700 of displaying ranked healthcare products. In an embodiment of the invention, the kiosk 160 executes the method 700. First, one or more symptoms (search terms) are received (710); then constraints are received (720). Based on the constraints, ingredients are filtered (730) out and healthcare products (results) that can relieve the systems and do not have filtered ingredients are determined (740). The determination (740) can be done using the algorithm discussed above or any other algorithm. The healthcare products are then ranked (750) based on bids and displayed (760) in order of their ranking. The ranking (750) for variable bids will be discussed in further detail in conjunction with FIG. 8 below. After the displaying (760), the advertiser that submitted the bid(s) is charged (770) accordingly. Healthcare products that are determined (740) to be appropriate can also be displayed in an unranked order (e.g., randomly) or based on other factors (e.g., store brands first).

In an embodiment of the invention, the highest ranked product may be displayed (760) second or third instead of first as some consumers may have a distrust of a first displayed product in a ranked system. As such, displaying a product in second or third place may increase trust in the product. It is important to note that all products displayed will be appropriate for the symptoms entered, i.e., products (search results) are first determined to be appropriate and only the display order of the determined products is effected by the bidding. Bidding does not effect actual selection of a product to be displayed. In other words, search results are first determined, then it is determined if there are bids for ranking of any of the search results. The search results are then ranked and displayed according to bids.

Displaying can include ordered lists, banners, etc. In an embodiment of the invention, the method 700 can be performed in order other than that described above. Further, the receiving (720) constraints may be eliminated in an embodiment.

FIG. 8 is a flowchart illustrating a method 750 of determining highest bidders for variable bids. If bids are variable, then first the highest maximum bid is determined (810). Afterwards, the second highest maximum bid is determined (820). The highest bid is then calculated (830) as the second highest maximum bid plus an increment (e.g., $1). Method 750 is then repeated for any subsequent variable bids.

The foregoing description of the illustrated embodiments of the present invention is by way of example only, and other variations and modifications of the above-described embodiments and methods are possible in light of the foregoing teaching. For example, while embodiments of the invention are used for the searching and ranking of healthcare products, it can be used for the searching and ranking of anything. Further, any search algorithm can be used. Although the network sites are being described as separate and distinct sites, one skilled in the art will recognize that these sites may be a part of an integral site, may each include portions of multiple sites, or may include combinations of single and multiple sites. Further, components of this invention may be implemented using a programmed general purpose digital computer, using application specific integrated circuits, or using a network of interconnected conventional components and circuits. Connections may be wired, wireless, modem, etc. The embodiments described herein are not intended to be exhaustive or limiting. The present invention is limited only by the following claims.

What is claimed is:

1. A computer-based method, comprising:
receiving a symptom;
determining healthcare products for treating the symptom;
ranking the determined products based on bids; and
displaying the determined products based on the ranking.

2. The method of claim 1, further comprising:
receiving a constraint; and
wherein the determining is further based on the received constraint.

3. The method of claim 2, wherein the constraint is an allergy.

4. The method of claim 2, wherein the constraint is an age restriction.

5. The method of claim 1, wherein the determining uses an ontological search method.

6. The method of claim 1, wherein the displaying displays the highest ranked product outside of a first position.

7. The method of claim 1, wherein the displaying displays a store brand product in a reserved position.

8. The method of claim 1, wherein the bidding is restricted to a specified geography.

9. The method of claim 1, wherein the bidding is time restricted.

10. The method of claim 1, wherein the bidding is variable priced.

11. A system, comprising:
a GUI capable of receiving a symptom;
a search agent, communicatively coupled to the GUI, capable of determining healthcare products for treating the symptom;
a ranking engine, communicatively coupled to the search agent, capable of ranking the determined products based on bids; and
wherein the GUI is further capable of displaying the determined products based on the ranking.

12. The system of claim 11, wherein:
the GUI is further capable of receiving a constraint; and
the search agent is further capable of determining based on the received constraint.

13. The system of claim 12, wherein the constraint is an allergy.

14. The system of claim 12, wherein the constraint is an age restriction.

15. The system of claim 11, wherein the search agent uses an ontological search method.

16. The system of claim 11, wherein the GUI displays the highest ranked product outside of a first position.

17. The system of claim 11, wherein the GUI displays a store brand product in a reserved position.

18. The system of claim 1, wherein the bidding is restricted to a specified geography.

19. The system of claim 1, wherein the bidding is time restricted.

20. The system of claim 1, wherein the bidding is variable priced.

21. A system, comprising:
means for receiving a symptom;
means for determining healthcare products for treating the symptom;
means for ranking the determined products based on bids; and
means for displaying the determined products based on the ranking.

22. A computer-readable medium having stored thereon instructions to cause a computer to execute a method, the method comprising:
receiving a symptom;
determining healthcare products for treating the symptom;
ranking the determined products based on bids; and
displaying the determined products based on the ranking.
